(19) United States
(12) Patent Application Publication
(10) Pub. No.: US 2013/0230894 A1
(43) Pub. Date: Sep. 5, 2013

(54) Optimised Fermentation Media

(71) Applicant: LANZATECH NEW ZEALAND LIMITED, Roselle, IL (US)

(72) Inventors: Christophe Daniel Mihalcea, Auckland (NZ); Jennifer Mon Yee Fung, Boston, MA (US); Bakir Al-Sinawi, Auckland (NZ); Loan Phuong Tran, Auckland (NZ)

(73) Assignee: LanzaTech New Zealand Limited, Roselle, IL (US)

(21) Appl. No.: 13/802,320

(22) Filed: Mar. 13, 2013

Related U.S. Application Data
(63) Continuation of application No. 13/058,393, filed on Aug. 8, 2011.
(60) Provisional application No. 61/172,783, filed on Apr. 26, 2009.
(60) Provisional application No. 61/119,021, filed on Dec. 1, 2008.
(60) Provisional application No. 61/119,018, filed on Dec. 1, 2008.

(51) Int. Cl. C12P 7/08

(52) U.S. Cl. CPC C12P 7/08
USPC 435/158; 435/163

(57) ABSTRACT

The invention relates to improvements in the production of alcohols by microbial fermentation, particularly to production of alcohols by microbial fermentation of a substrate comprising CO. It more particularly relates to the addition of an inorganic sulfur source to a fermentation system such that one or more micro-organisms convert a substrate comprising CO to alcohols. In a particular embodiment, a microbial culture is provided with sodium polysulfide, wherein a substrate comprising CO is converted to products including ethanol and 2,3-butanediol.

### CROSS REFERENCE TO RELATED APPLICATIONS

This application is a continuation of copending U.S. application Ser. No. 13/058,393 filed on Aug. 8, 2011; which is a National Stage of International Application No. PCT/NZ2009/000268 filed on Nov. 30, 2009, which claims the benefit of the priority dates of the following US Provisional Applications: U.S. Provisional Application No. 61/119,018 filed Dec. 1, 2008; U.S. Provisional Application 61/119,021 filed Dec. 1, 2008 and U.S. Provisional Application 61/172,783 filed Apr. 26, 2009. The contents of all of the prior applications mentioned above are incorporated herein by reference in their entirety.

### FIELD OF THE INVENTION

This invention relates generally to methods for increasing the efficiency of microbial growth and production of products by microbial fermentation on gaseous substrates. More particularly the invention relates to processes for producing alcohols, particularly ethanol, by microbial fermentation of gases containing carbon monoxide.

### BACKGROUND OF THE INVENTION

Ethanol is rapidly becoming a major hydrogen-rich liquid transport fuel around the world. Worldwide consumption of ethanol in 2005 was an estimated 12.2 billion gallons. The global market for the fuel ethanol industry has also been predicted to continue to grow sharply in future, due to an increased interest in ethanol in Europe, Japan, the USA and several developing nations.

For example, in the USA, ethanol is used to produce E10, a 10% mixture of ethanol in gasoline. In E10 blends, the ethanol component acts as an oxygenating agent, improving the efficiency of combustion and reducing the production of air pollutants. In Brazil, ethanol satisfies approximately 30% of the transport fuel demand, as both an oxygenating agent blended in gasoline, or as a pure fuel in its own right. Also, in Europe, environmental concerns surrounding the consequences of Green House Gas (GHG) emissions have been the stimulus for the European Union (EU) to set member nations a mandated target for the consumption of sustainable transport fuels such as biomass derived ethanol.

The vast majority of fuel ethanol is produced via traditional yeast-based fermentation processes that use crop derived carbohydrates, such as sucrose extracted from sugarcane or starch extracted from grain crops, as the main carbon source. However, the cost of these carbohydrate feed stocks is influenced by their value as human food or animal feed, and the cultivation of starch or sucrose-producing crops for ethanol production is not economically sustainable in all geographies. Therefore, it is of interest to develop technologies to convert lower cost and/or more abundant carbon resources into fuel ethanol.

CO is a major free energy-rich by-product of the incomplete combustion of organic materials such as coal or oil and oil derived products. For example, the steel industry in Australia is reported to produce and release into the atmosphere over 500,000 tonnes of CO annually.

Catalytic processes may be used to convert gases consisting primarily of CO and/or CO and hydrogen (H₂) into a variety of fuels and chemicals. Micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. While using CO as the sole carbon source all such organisms produce at least two of these end products.

Anaerobic bacteria, such as those from the genus Clostridium, have been demonstrated to produce ethanol from CO, CO₂ and H₂ via the acetyl CoA biochemical pathway. For example, various strains of Clostridium ljungdahlii that produce ethanol from gases are described in WO 00/68407, EP 117309, U.S. Pat. Nos. 5,173,429, 5,593,886, and 6,368,819, WO 98/00558 and WO 02/08438, each of which is incorporated herein by reference. The bacterium Clostridium autoethanogenum sp is also known to produce ethanol from gases (Abrini et al, Archives of Microbiology 161, pp 345-351 (1994)).

However, ethanol production by micro-organisms by fermentation of gases is always associated with co-production of acetate and/or acetic acid. As some of the available carbon is converted into acetate/acetic acid rather than ethanol, the efficiency of production of ethanol using such fermentation processes may be less than desirable. Also, unless the acetate/acetic acid by-product can be used for some other purpose, it may pose a waste disposal problem. Acetate/acetic acid is converted to methane by micro-organisms and therefore has the potential to contribute to GHG emissions.

Several enzymes known to be associated with the ability of micro-organisms to use carbon monoxide as their sole source of carbon and energy are known to require metal co-factors for their activity. Examples of key enzymes requiring metal cofactor binding for activity include carbon monoxide dehydrogenase (CODH), and acetyl—CoA synthase (ACS).

WO2007/117157, WO2008/115080, WO2009/022925, WO2009/058028, WO2009/064200, WO2009/064201 and WO2009/113878, the disclosure of which are incorporated herein by reference, describe processes that produce alcohols, particularly ethanol, by anaerobic fermentation of gases containing carbon monoxide. Acetate produced as a by-product of the fermentation process described in WO2007/117157 is converted into hydrogen gas and carbon dioxide gas, either or both of which may be used in the anaerobic fermentation process. WO2009/022925 discloses the effect of pH and ORP in the conversion of substrates comprising CO to products such as acids and alcohols by fermentation. WO2009/058028 describes the use of industrial waste gases for the production of products, such as alcohol, by fermentation. WO2009/064201 discloses carriers for CO and the use of CO in fermentation. WO2009/113878 discloses the conversion of acid(s) to alcohol(s) during fermentation of a substrate comprising CO.

Microbes capable of growing on CO-containing gases are known to do so at a slower rate than is traditionally associated with microbes grown on sugars. From a commercial perspective, in a fermentation process the time required for a microbial population to grow to a sufficiently high cell density to allow an economically viable level of product to be synthesised, is a key operating cost affecting the profitability of the process. Technologies that act to enhance culture growth rates and therefore reduce the time required for cell populations to reach high cell densities may serve to improve the commercial viability of the overall process.

In fermentation processes dedicated to the production of alcohols from gas feedstocks, ensuring that the appropriate supplements (i.e., nutrients) are present in the correct concentrations and in readily bioavailable forms, may be critical to maintaining optimal microbial growth and/or alcohol productivities. For example, sulfur is typically provided in the form of cysteine or sulfide to provide a sulfur source necessary to support enzymatic processes occurring in a microbial culture. Sulfur sources such as sulfide exist in equilibrium with hydrogen sulfide in typical fermentation media. Hydrogen sulfide has limited solubility in water and the concentration of sulfur in solution can be significantly reduced where gaseous substrates are sparged through the fermentation media. Accordingly, identification of an improved or alternative sulfur source required for alcohol production and the threshold concentrations required in fermentation systems using carbon monoxide containing gases as a feedstock, is a key component in ensuring high alcohol production rates and low process operating costs.

Sulfur sources such as cysteine and/or sulfide are also used to attain desirable ORP of the anaerobic fermentation broth prior to inoculation. However, such reducing agents are slow and have limited reducing power. Furthermore, when these sulfur containing compounds are used to reduce ORP of a fermentation media, they are oxidised themselves. For example, cysteine is oxidised to the dimer cysteine. It is considered that the reduced form of these compounds is substantially more bioavailable as a sulphur source for consumption by a microbial culture than the oxidised form. As such, when a sulphur source is used to lower the ORP of a fermentation reaction, the actual concentration of sulfur available to the microbial culture will decrease. Accordingly, identification of an improved or alternative reducing agent for use with anaerobic fermentation systems using carbon monoxide containing gases as a feedstock, is a key component in ensuring high alcohol production rates and low process operating costs.

It is an object of the present invention to provide a process that goes at least some way towards overcoming the above disadvantages, or at least to provide the public with a useful choice.

### SUMMARY OF THE INVENTION

In one broad aspect, the invention provides a method of improving growth efficiency of a bacterial culture, the method comprising the step of adding an inorganic sulfur source to the culture.

In a second broad aspect, there is provided a method of maintaining or increasing production rates of one or more products produced by a microbial culture, the method comprising the step of adding an inorganic sulfur source to the culture.

In particular embodiments of the first two aspects, the inorganic sulfur source comprises one or more inorganic sulfur compounds and is adapted to release one or more sulfur containing species into a fermentation broth. In particular embodiments, the one or more sulfur containing species can be utilised by a microbial culture.

In particular embodiments of the first two aspects, the method comprises maintaining the concentration of one or more inorganic sulfur compounds at approximately 0.1 mM to 20 mM sulfur, or about 1 mM to 20 mM sulfur.

In another broad aspect, the present invention provides a method of producing one or more acid and/or alcohol by microbial fermentation, the method comprising the steps of:
(a) providing a substrate comprising carbon monoxide and optionally carbon dioxide and/or hydrogen;
(b) in a bioreactor containing a culture of one or more micro-organisms anaerobically fermenting the substrate to produce one or more products consisting of acids and/or alcohols;
(c) capturing and recovering the one or more products produced;
wherein the concentration of one or more inorganic sulfur compounds in the culture is kept within the range of about 1 mM to about 20 mM sulfur during at least step (b).

In another broad aspect, the invention provides a method of improving growth efficiency and/or maintaining or increasing productivity of one or more acid and/or alcohol by microbial fermentation, the method comprising the steps of:
(a) providing a substrate comprising CO;
(b) in a bioreactor containing a culture of one or more micro-organisms in a liquid nutrient medium, anaerobically fermenting the substrate to produce one or more products consisting acids and/or alcohols;
(c) capturing and recovering the one or more products produced;
   wherein the liquid nutrient medium comprises one or more inorganic sulfur compound.

In one embodiment, the concentration of one or more inorganic sulfur compounds in the liquid nutrient medium is provided in a desired concentration range of about 1 mM to about 20 mM sulfur. In certain embodiments, the one or more inorganic sulfur compound is provided in a desired concentration range of about 1 mM to about 20 mM sulfur per gram of cells. In one embodiment, the one or more inorganic sulfur compound is provided in a desired concentration range of about 6 mM to about 18 mM sulfur.

In another broad aspect, the invention provides a method of improving growth efficiency and/or maintaining or increasing productivity of one or more acid and/or alcohol by microbial fermentation, the method comprising the steps of:
(a) providing a substrate comprising CO;
(b) in a bioreactor containing a culture of one or more micro-organisms in a liquid nutrient medium comprising one or more inorganic sulfur compound, anaerobically fermenting the substrate to produce one or more products consisting acids and/or alcohols;
(c) capturing and recovering the one or more products produced;
   wherein the one or more inorganic sulfur compound is adapted to release one or more sulfur containing species into the liquid nutrient medium.

In certain embodiments, the method includes:
(a) taking a sample of the liquid nutrient medium;
(b) determining the concentration of sulfur, metal(s), alcohol and/or acids concentration and/or microbial cell density;
   wherein the predetermined amount required to maintain the sulfur and/or metal(s) within the desired range may be calculated from the sulfur concentration, metal(s) concentration, alcohol and/or acid concentration and/or microbial cell density.

According to another broad aspect, there is provided a method of producing products including alcohol and optionally acetate, the method including the steps of:
(a) providing a substrate comprising CO;
(b) in a bioreactor containing a culture of one or more micro-organisms in a liquid nutrient medium comprising one or more inorganic sulfur compound, anaerobically fermenting the substrate to produce one or more products consisting acids and/or alcohols;
   wherein the alcohol to acetate ratio is at least 10:1. In particular embodiments, the alcohol to acetate ratio is at least 20:1; or at least 50:1; or at least 100:1.

According to another broad aspect, there is provided a method of producing alcohol by microbial fermentation, wherein the alcohol is produced without concomitant acetate production. In certain embodiments, the microbial fermentation includes:
(a) providing a substrate comprising CO;
(b) in a bioreactor containing a culture of one or more micro-organisms in a liquid nutrient medium and anaerobically fermenting the substrate to produce alcohol.

In particular embodiments, the method includes adding one or more inorganic sulfur compound to the microbial fermentation. In particular embodiments, the method includes adding one or more inorganic sulfur compound to the microbial fermentation, such that alcohol is produced without concomitant acid production.

In particular embodiments of the various aspects, the one or more inorganic sulfur compound is adapted to release hydrogen sulfide and/or sulfide into the liquid nutrient medium. In particular embodiments, the hydrogen sulfide is released at a rate such that hydrogen sulfide concentration in the liquid nutrient medium is substantially maintained above a desired level. In certain embodiments, the hydrogen sulfide is substantially maintained above a desired level sufficient to promote growth and/or alcohol productivity. In particular embodiments, the one or more inorganic sulfur compound is adapted to release polysulfide into the liquid nutrient media. In particular embodiments, the one or more inorganic sulfur compound is adapted to provide a colloidal sulfur dispersion in the liquid nutrient medium. In particular embodiments, the one or more inorganic sulfur compound is provided such that the sulfur species, such as polysulfide and/or sulfide and/or colloidal sulfur is maintained at a desirable concentration to promote growth and/or alcohol productivity. In particular embodiments, the one or more inorganic sulfur compound is polysulfide. It is recognised that in such embodiments, the polysulfide will decompose to sulfur species such as polysulfide chains of varying “S” chain length, sulfide, colloidal sulfur and/or elemental sulfur.

In particular embodiments of the various aspects, the one or more inorganic sulfur compound comprises polysulfide. In particular embodiments, polysulfide is provided such that the concentration in the liquid nutrient medium is maintained at about 1 mM to about 20 mM sulfur. In particular embodiments, the polysulfide is provided such that the concentration in the liquid nutrient medium is maintained at about 6 mM. In certain embodiments, the polysulfide is provided in the form of sodium polysulfide.

In particular embodiments, the method comprises the step of maintaining the concentration of one or more of manganese, zinc, copper, boron, molybdenum, selenium, vanadium, iron, cobalt and nickel within the following desired ranges:

| | |
|---|---|
| Iron | about 0.3 mM to about 5 mM |
| Zinc | about 7 μM to about 0.5 mM |
| Manganese | about 30 μM to about 0.5 mM |
| Boron | about 0.1 mM to about 0.5 mM |
| Cobalt | about 17 μM to about 2.5 mM |
| Nickel | about 3 μM to about 2.5 mM |
| Molybdenum | about 2.75 μM to about 0.5 mM |
| Selenium | about 1.2 μM to about 0.5 mM |
| Tungsten | about 6 μM to about 0.5 mM |
| Vanadium | about 10 μM to about 1 mM |

In particular embodiments, the one or more metals are provided at the above concentrations per gram of cells.

In some embodiments, the concentration of sulfur and/or metal(s) are kept within the desired range by adding a predetermined amount of one or more inorganic sulfur compound and/or metal salt(s) to the culture continuously and/or at predetermined time intervals.

In a further aspect, the invention provides a nutrient media adapted to improve microbial growth and/or maintain or improve productivity of a microbial culture, the media comprising one or more inorganic sulfur compound. In a particular embodiment, the medium comprises one or more inorganic sulfur compound at a concentration of about 1 mM to about 20 mM sulfur.

In particular embodiments, the liquid nutrient medium further includes one or more of manganese, zinc, boron, molybdenum, selenium, vanadium, tungsten, iron, cobalt and nickel.

In some embodiments, the one or more inorganic sulfur compound and/or metal(s) are added to the liquid nutrient medium in the form of a salt. In particular embodiments, the one or more inorganic sulfur compound and/or metal(s) are added in the form of a composition comprising one or more inorganic sulfur compound and/or metal salt(s) in combination with one or more diluents and/or other ingredients.

In another broad aspect, there is provided a composition comprising one or more inorganic sulfur compound. In particular embodiments, the composition is added to a fermentation media, wherein one or more sulfur containing species, such as hydrogen sulfide and/or sulfide, is released at a predetermined rate. In particular embodiments, the hydrogen sulfide and/or sulfide are released at a rate such that hydrogen sulfide concentration in the liquid nutrient medium is substantially maintained above a desired level. In certain embodiments, the hydrogen sulfide is substantially maintained above a desired level sufficient to promote growth and/or alcohol productivity.

In another broad aspect, the invention provides a method of improving growth efficiency of a microbial culture in anaerobic fermentation, the method comprising the step of adding a metal reducing agent to the culture.

In another broad aspect, the invention provides a method of maintaining or increasing production rates of one or more products produced by a microbial culture in anaerobic fermentation, the method comprising the step of adding a metal reducing agent to the culture.

In another broad aspect, the invention provides a method of decreasing redox potential of a microbial culture in anaerobic fermentation, the method including the step of adding one or more metal reducing agents to the microbial culture.

In a further broad aspect, the invention provides a method of attaining and/or maintaining a desirable redox potential of a microbial culture in anaerobic fermentation, the method including the step of adding one or more metal reducing agents to a fermentation broth containing the microbial culture.

In particular embodiments of the previous aspects, the method includes adding one or more metal reducing agents to a fermentation broth containing the microbial culture, such that the redox potential of the broth decreases. In particular embodiments, the metal reducing agent is added to reduce the redox potential of the fermentation broth to a desirable level to support growth and/or production of one or more products. In certain embodiments, the redox potential is reduced to less than −200 mV, or less than −250 mV, or less than −300 mV, or less than −350 mV, or less than −400 mV, or less than −450 mV, or less than −500 mV.

In some embodiments, the metal reducing agent may be added substantially continuously or on an as required basis to maintain the redox potential at a desirable level. The redox potential of the broth can be monitored by means known in the art, such as an ORP probe. In particular embodiments, the one or more metal reducing agents can be added when the ORP of the fermentation broth increases above a predetermined threshold.

In another aspect, there is provided a method of decreasing redox potential of a liquid nutrient medium prior to inoculation with a microbial culture, the method including the step of adding one or more metal reducing agents to the liquid nutrient medium.

In particular embodiments, the redox potential of the liquid nutrient medium can be reduced to a desirable level sufficient to support microbial growth following inoculation. In certain embodiments, the redox potential of the liquid nutrient medium is reduced to less than −150 mV, or −200 mV, or less than −250 mV, or less than −300 mV, or less than −350 mV, or less than −400 mV, or less than −450 mV, or less than −500 mV.

In particular embodiments according to the various aspects, the anaerobic fermentation is the fermentation of a substrate comprising CO.

In particular embodiments of the various aspects, the substrate comprising CO is gaseous. In particular embodiments, the gaseous substrate comprises a gas obtained as a by-product of an industrial process. In certain embodiments, the industrial process is selected from the group consisting of ferrous metal products manufacturing, non-ferrous products manufacturing, petroleum refining processes, gasification of biomass, gasification of coal, electric power production, carbon black production, ammonia production, methanol production and coke manufacturing. In a particular embodiment, the gaseous substrate comprises a gas obtained from a steel mill.

In certain embodiments, the CO-containing substrate will typically contain a major proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

In particular embodiments, the alcohol produced by the fermentation process is ethanol. The fermentation reaction may also produce acetate.

In particular embodiments, the fermentation reaction is carried out by one of more strains of acetogenic bacteria. Preferably, the acetogenic bacterium is selected from Clostridium, Moorella and Carboxydothermus, such as Clostridium autoethanogenum, Clostridium ljungdahlii and Morella thermoacetica. In one embodiment, the acetogenic bacterium is Clostridium autoethanogenum.

Although the invention is broadly as defined above, it is not limited thereto and also includes embodiments of which the following description provides examples.

### BRIEF DESCRIPTION OF THE DRAWINGS

The invention will now be described in detail with reference to the accompanying Figures in which:

FIG. 1A: Metabolite production and microbial growth in batch fermentation of CO by C. auto in presence of polysulfide.

FIG. 1B: Metabolite production and microbial growth in batch fermentation of CO by C. auto in presence of polysulfide.

FIG. 1C: Metabolite production and microbial growth in batch fermentation of CO by C. auto in presence of polysulfide.

FIG. 2: Metabolite production and microbial growth in batch fermentation of CO by C. auto in presence of polysulfide.

FIG. 3: Metabolite production and microbial growth in batch fermentation of CO by C. auto in presence of polysulfide.

FIG. 4: Metabolite production and microbial growth in batch fermentation of CO by C. auto in presence of polysulfide, wherein precipitate is removed prior to inoculation.

FIG. 5: Metabolite production and microbial growth in batch fermentation of CO by C. auto in presence of polysulfide, wherein metal sulfide precipitate is added prior to inoculation.

FIG. 6: Metabolite production and microbial growth in batch fermentation of CO by Clostridium autoethanogenum in the presence of polysulfide.

### DETAILED DESCRIPTION OF THE INVENTION

In accordance with particular embodiments of the invention, an inorganic sulfur source comprising one or more inorganic sulfur compound(s) is added to support growth and/or productivity of a microbial culture producing products such as alcohols and/or acids by anaerobic fermentation of carbon monoxide. As noted previously, cysteine is typically used to provide the sulfur necessary to support growth and/or productivity of the microbial culture. However, it has been surprisingly recognised that an inorganic sulfur source can be used instead of cysteine. In particular embodiments of the invention, the one or more inorganic sulfur compound(s) provided to the microbial culture improves growth rates and overall microbial cell accumulation in comparison to cysteine supplied cultures. Furthermore, the one or more inorganic sulfur compound(s) maintain or improve the productivity of alcohols by the culture. In particular embodiments of the invention, the inorganic sulfur source is adapted to release one or more sulfur containing species into a fermentation broth, such that the one or more sulfur containing species can be utilised by the microbial culture.

It is well known that sulfur is required in various enzymatic processes within many microbial cultures. For example, sulfur (partially in the form of cysteine) comprises a major component of the electron transfer mediator ferredoxin. However, when cysteine is provided in typical concentrations (e.g. 0.25 g/L U.S. Pat. No. 7,285,402), the microbial culture may be sulfur limited and unable to achieve high growth and product synthesis efficiency. Furthermore, when sulfur is provided in the form of a simple sulfide salt such as Na₂S, the resulting hydrogen sulfide concentration in the fermentation broth may decrease over time due to evaporation.

In particular embodiments of the invention, an inorganic sulfur source is provided, such that when the inorganic sulfur source is added to an aqueous nutrient media, one or more sulfur containing species are produced and are available to support microbial growth and/or product production. In particular embodiments, the sulfur containing species is at least one polysulfide species (such as Sₓ²⁻) and/or a sulfide species (such as HS⁻, S²⁻) and/or colloidal sulfur.

In accordance with the methods of the invention, one or more inorganic sulfur compound(s) such as polysulfide can be supplied to a microbial culture without causing detrimental effect, resulting in increased growth and/or alcohol productivity and potentially dramatically reducing operating costs. Without wishing to be bound by theory, it is considered that while sulfur is generally provided to and utilised by biological systems in the form of cysteine, sulfur (in the form of one or more sulfur containing species) obtained from one or more inorganic sulfur compound(s) is substantially more bioavailable to micro-organisms that produce products by anaerobic fermentation of carbon monoxide.

In accordance with particular methods of the invention, there is provided an inorganic sulfur source adapted to release one or more sulfur containing species, which may be utilised by a microbial culture, substantially continuously over a time period. Formally, polysulfide (Sₓ²⁻ or HSₓ⁻) consists of a sulfide anion and a chain of zerovalent sulfur atoms, S(0). Without wishing to be bound by theory, the predominant polysulfide species at pH>6 appear to be S₄²⁻ and S₅²⁻, in aqueous solutions. However, the different polysulfide species cannot be quantified directly, since they disproportionate rapidly in aqueous solutions into chain lengths ranging from S₂ to about S₉, for example:

4S₄²⁻+H⁺ [symbol] 3S₅²⁻+HS⁻

Upon addition of polysulfide to water, the resulting mixture will be in equilibrium with elemental sulfur (or colloidal sulfur) and sulfide as follows:

S₉²⁻+H₂O [symbol] S₈+HS⁻+OH⁻

Accordingly, in particular embodiments, the microbial culture utilises colloidal sulfur or elemental sulfur dispersed in the liquid nutrient medium. In particular embodiments, when polysulfide is added to fermentation media at pH5.5, a white dispersion forms. Without wishing to be bound by theory, it is considered that the white dispersion comprises colloidally suspended sulfur. As such, in particular embodiments, the sulfur containing species utilised by the microbial culture is a sulfur dispersion that contains emulsified sulfur, obtained under reducing conditions. In particular embodiments, the colloidal sulfur has some sulfonic characteristics. Additionally, or alternatively, the colloidal sulfur has some sulfidic characteristics. In particular embodiments, the sulfur dispersion is a sulfur sol. Sols are dispersions of very small sulfur droplets in aqueous media and are typically hydrophilic or hydrophobic. The types of sols formed upon decomposition of polysulfide in an aqueous solution are described in Top. Curr. Chem. (2003) 230: 153-166, which is incorporated herein by reference. In one exemplary method of the invention, a white sulfur sol is produced when polysulfide is added to aqueous media at pH 5.5.

In particular embodiments, the sulfur containing species utilised by the microbial culture is HSₓ⁻ and/or Sₓ²⁻, wherein x>1. In particular embodiments, the one or more sulfur containing species is released in aqueous nutrient media such that the concentration of the sulfur containing species in the media is maintained above a level sufficient to promote growth and/or alcohol productivity. In one exemplary method of the invention, sodium polysulfide is added to the nutrient media to promote growth and/or alcohol productivity. The types of sulfur species formed when polysulfide is dissolved in an aqueous solution are detailed in Top. Curr. Chem. (2003) 231:127-152, which is incorporated herein by reference, and include polysulfide ions of various chain lengths, colloidal sulfur and sulfides, such as hydrogen sulfide.

In particular embodiments, the sulfur containing species that can be utilised by the microbial culture is elemental sulfur. In particular embodiments, the sulfur containing species that can be utilised by a microbial culture is hydrogen sulfide and/or sulfide. In an aqueous system, hydrogen sulfide exists in equilibrium with sulfide and dihydrogen sulfide:

H₂S [symbol] HS⁻ [symbol] S²⁻

However, in acidic media, the amount of sulfide (S²⁻) will be very minor.

According to particular embodiments of the invention, polysulfide can be added to a fermentation broth such that sulfur containing species is substantially continuously available over a time period as polysulfide disproportionates. In particular embodiments, hydrogen sulfide and/or sulfide are released at a rate such that hydrogen sulfide concentration in the liquid nutrient medium is substantially maintained above a desired level or within a desired concentration range. In certain embodiments, the hydrogen sulfide and/or sulfide is substantially maintained above a desired level sufficient to promote growth and/or alcohol productivity.

According to some embodiments of the invention, dissolved hydrogen sulfide will precipitate as one or more metal sulfides. The precipitated sulfide(s) may be sparingly soluble and thus maintain a sulfide concentration in the liquid nutrient medium above a desired level.

According to some embodiments, there is provided a composition comprising one or more inorganic sulfur compound. In particular embodiments, the composition is added to a fermentation media, wherein one or more sulfur species is released at a predetermined rate. In particular embodiments, colloidal sulfur is released at a predetermined rate, such that the colloidal sulfur is substantially maintained above a desired level. In particular embodiments, hydrogen sulfide and/or sulfide are released at a rate such that hydrogen sulfide concentration in the liquid nutrient medium is substantially maintained above a desired level. In certain embodiments, the colloidal sulfur and/or hydrogen sulfide and/or sulfide is substantially maintained above a desired level sufficient to promote growth and/or alcohol productivity.

In particular embodiments, the inorganic sulfur source is optionally provided in a novel nutrient media comprising at least one of manganese, zinc, boron, molybdenum, selenium, vanadium, iron, cobalt, tungsten and nickel. In accordance with particular embodiments of the invention, manganese, zinc, copper, boron, molybdenum, selenium, vanadium, iron, cobalt and nickel can be depleted during growth of Clostridium autoethanogenum on a gaseous substrate comprising carbon monoxide. Furthermore, these metals can be depleted during the production of alcohols. Whilst not wishing to be bound by any particular theory, it is considered the depletion of one or more of these metals represents a rate limiting factor in the growth of Clostridium autoethanogenum on carbon monoxide, and concomitantly, product biosynthesis and/or productivity. The growth rate of Clostridium autoethanogenum and thus the efficiency of fermentation reactions involving Clostridium autoethanogenum may be increased by adding these metals at certain time points during the fermentation process. In accordance with the invention, there is provided a novel nutrient media in which these metals are provided in higher concentrations, to help prevent depletion to a rate limiting point. The invention has significant benefit in maintaining or ensuring adequate, preferably high, microbial growth rates and alcohol productivity, increasing efficiency of fermentation processes and lowering process operating costs.

As noted above, in some embodiments of the invention, the inorganic sulfur source releases hydrogen sulfide in the nutrient media and may be consumed by the microbial culture. Excess hydrogen sulfide not consumed by the culture may be, at least in part, evaporated and may exit a bioreactor as an exhaust gas. Additionally, or alternatively, excess sulfide may, at least in part, react with one or more of manganese, zinc, copper, boron, molybdenum, selenium, vanadium, iron, cobalt and nickel to form metal sulfide(s). Such sulfides are generally poorly soluble in water and tend to precipitate from solution.

In accordance with particular methods of the invention, precipitates comprising one or more metal sulfides are formed in and/or added to the liquid nutrient medium. Such precipitates form a protective environment for the microbial culture to grow and/or produce metabolites, such as acids and/or alcohols. As such, in particular embodiments of the invention, a metal sulfide precipitate is provided such that microbial growth and/or metabolite production is improved. Without wishing to be bound by theory, it is considered that during times of stress, such as the period following inoculation, a microbial culture may produce hydrogen sulfide. It is further considered that provision of a metal sulfide precipitate effectively traps the hydrogen sulfide by providing nucleation sites, thus making the sulfide substantially unavailable to the microbial culture. It is also considered that removing hydrogen sulfide formed by the culture improves the overall efficiency of the fermentation.

Additionally or alternatively, the metals interact with sulfidic sulfur sols to stabilise the dispersion. In particular embodiments, the metal-sol species are utilised by the microbial culture and improve growth and/or production of products such as alcohols.

In accordance with the methods of the invention, the one or more inorganic sulfur compound is added to a microbial culture such that a substrate comprising CO is converted to products including alcohols such as ethanol and/or 2,3-butanediol. Surprisingly, addition of inorganic sulfur to fermentation media can enhance microbial growth and/or alcohol production. Typically, alcohol, such as ethanol is produced concomitantly with acids, such as acetate, during fermentation of CO. However, in accordance with the methods of the invention, the alcohol to acid product ratio is at least 10:1; or at least 20:1; or at least 50:1; or at least 100:1 favouring alcohol. In particular embodiments, alcohol is produced without concomitant acids production.

In particular embodiments, 2,3-butandiol is produced in addition to ethanol. For example, fermentation of a substrate comprising CO by C. auto, ethanol and 2,3-butanediol are typically produced in a ratio of at least 10:1 favouring ethanol. However, in accordance with particular methods of the invention, 2,3-butanediol is provided such that the ethanol:2,3-butanediol ratio is less than 10:1, or less than 8:1, or less than 5:1, or less than 3:1, or less than 2:1.

In accordance with particular embodiments of the invention, one or more metal reducing agent(s) is provided to a liquid nutrient media and/or fermentation broth to attain and/or maintain the ORP of a microbial culture at a sufficient level to support growth and/or alcohol productivity. It is well known that sulfur compounds such as cysteine and sodium sulfide can be partially oxidised to attain and/or maintain a desirable ORP sufficient to support microbial growth and/or alcohol production in a microbial culture by fermentation of CO. However, it is considered that the partially oxidised sulfur compound (e.g. cysteine) is less bioavailable to the microbial culture as an essential source of sulphur. In addition, when cysteine is used as a reducing agent, the ORP of an aqueous fermentation media can only be decreased to around −150 to −250 mV and this decrease is very slow (e.g., can take several hours).

It has been surprisingly found that a metal reducing agent, such as Cr(II), can be used to quickly reduce the ORP of an aqueous fermentation media to a desirable level suitable to support growth of an anaerobic micro-organism. In accordance with particular methods of the invention, the reducing agent, such as Cr(II), can be added prior to inoculation to attain an anaerobic environment with an ORP sufficiently low to support microbial growth. For example, Cr(II) can be used to reduce the ORP of an aqueous fermentation media to less than −150 mV, or less than −200 mV, or less than −250 mV, or less than −300 mV, or less than −350 mV, or less than −400 mV, or less than −450 mV, or less than −500 mV. Additionally or alternatively, the metal reducing agent, such as Cr(II), can be added to the fermentation broth while the microbial culture is growing and/or producing products to maintain a media ORP sufficiently low to promote desirable product production. For example, Cr(II) can be used to reduce the ORP of a fermentation broth to less than −200 mV, or less than −250 mV, or less than −300 mV, or less than −350 mV, or less than −400 mV, or less than −450 mV, or less than −500 mV. Accordingly, when one or more essential nutrients, such as a source of sulfur (e.g., cysteine), is added to the microbial culture, it will remain substantially unoxidised and thus more available for consumption by the microbial culture.

It is recognised that while pH can be adjusted to maintain media ORP within a preferred range according to the Nernst equation, it is typically desirable to maintain the pH of a fermentation broth substantially constant for optimal growth and/or alcohol productivity. For example, the optimal pH for Clostridium autoethanogenum is 5.0-6.5.

In accordance with particular embodiments of the invention, predetermined amounts of metal reducing agents, such as Cr(II) can be added to a fermentation broth to rapidly attain a desired ORP. For example, a concentrated aqueous solution (such as 0.1-1.0M) of one or more metal reducing agents can be injected into a bioreactor to rapidly lower the ORP of the fermentation broth to a desired level. In accordance with particular aspects of the invention, Cr(II) is used, as it is one of the fastest known metal ions to react with oxygen and lower the ORP of a solution.

Use of one or more metal reducing agents also has further advantages. It is well known that acetic acid produced by a microbial culture can be reduced to ethanol by lowering the redox potential of the fermentation broth. An ORP sufficiently lower than the natural state of the culture causes microbial NAD(P)H to be in abundance and promote the reduction of acetic acid to ethanol by the microbial culture. A metal reducing agent, such as Cr(II) can be used to assist lowering the ORP of a microbial culture to less than −400 mV, or less than −450 mV, or less than −500 mV, or less than −550 mV, or less than −600 mV.

Without wishing to be bound by theory, while it may be possible for a microbial culture to attain and/or maintain a low ORP independently (for example, via oxidation of CO to CO₂), the use of one or more metal reducing agents conserves at least a portion of the cultures reducing power for the reduction of acetate to ethanol. In accordance with the methods of the present invention, a metal reducing agent (such as Cr(II)) can be used to support and/or increase alcohol productivity of a microbial culture by anaerobic fermentation of a substrate comprising CO.

The invention may be readily applicable to fermentation reactions which utilise substrates other than carbon monoxide, produce alcohols other than ethanol, and utilise micro-organisms other than Clostridium autoethanogenum.

### DEFINITIONS

Unless otherwise defined, the following terms as used throughout this specification are defined as follows:

The terms “increasing the efficiency”, “increased efficiency” and the like, when used in relation to a fermentation process, include, but are not limited to, increasing one or more of: the rate of growth of micro-organisms catalysing the fermentation, the volume of desired product (such as alcohols) produced per volume of substrate (such as carbon monoxide) consumed, the rate of production or level of production of the desired product, and the relative proportion of the desired product produced compared with other by-products of the fermentation.

The term “substrate comprising carbon monoxide” and like terms should be understood to include any substrate in which carbon monoxide is available to one or more strains of bacteria for growth and/or fermentation, for example.

“Gaseous substrate comprising carbon monoxide” include any gas which contains carbon monoxide. The gaseous substrate will typically contain a significant proportion of CO, preferably at least about 5% to about 100% CO by volume.

The term “co-substrate” refers to a substance that while not being the primary energy and material source for product synthesis, can be utilised for product synthesis when added in addition to the primary substrate.

The use of term “acid”, “acids” and the like when referring to adding an “acid” to a culture or bioreactor in accordance with the invention should be taken broadly, including any monocarboxylic and dicarboxylic acids. In addition reference to addition of “acids(s)” should be taken to include reference to the equivalent salt or a mixture of salt and acid. Similarly, references to specific acids herein should be taken to include reference to equivalent salts (for example butyric acid and butyrate) and vice versa. The ratio of molecular acid to carboxylate in the fermentation broth is dependent upon the pH of the system. Exemplary acids include acetic acid, propionic acid, n-butyric acid, n-pentanoic acid, n-hexanoic acid, and benzoic acid.

The term “bioreactor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Bubble Column, Gas Lift Fermenter, Membrane Reactor such as Hollow Fibre Membrane Bioreactor (HFMBR), Static Mixer, or other vessel or other device suitable for gas-liquid contact.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

The term “limiting concentration” means an initial concentration of a given component in a microbial fermentation medium that is sufficiently low to ensure that it will be depleted at some stage in the fermentation.

The term ORP and redox potential as used herein refers to the redox potential of an aqueous solution measured against Ag/Ag—Cl type electrode utilising a 3.8M KCl electrolyte salt bridge.

As will be appreciated from the description provided herein after, the phrase “metal reducing agent” and the like includes any metal, a metal salt or metal ion that can be used to reduce the ORP of an aqueous anaerobic fermentation media to −150 mV or less.

The invention provides methods for increasing the efficiency of microbial fermentation processes and methods for increasing the growth of micro-organisms used in the fermentation process. These methods involve utilising an improved nutrient or growth media in the fermentation reaction or supplementing the media during the fermentation process.

While the following description focuses on particular embodiments of the invention, namely the production of ethanol and/or acetate using CO as the primary substrate, it should be appreciated that the invention may be applicable to production of alternative alcohols and/or acids and the use of alternative substrates as will be known by persons of ordinary skill in the art to which the invention relates. For example, gaseous substrates containing carbon dioxide and hydrogen may be used. Further, the invention may be applicable to fermentation to produce butyrate, propionate, caproate, ethanol, propanol, and butanol. The methods may also be of use in producing hydrogen. By way of example, these products may be produced by fermentation using microbes from the genus Moorella, Clostridia, Ruminococcus, Acetobacterium, Eubacterium, Butyribacterium, Oxobacter, Methanosarcina, Methanosarcina, and Desulfotomaculum.

### Fermentation Reaction

The invention has particular applicability to supporting the production of ethanol from gaseous substrates such as high volume CO-containing industrial flue gases. In some embodiments of the invention, the substrate comprising CO is derived from carbon containing waste, for example, industrial waste gases or from the gasification of other wastes. As such, the methods of the invention represent effective processes for capturing carbon that would otherwise be exhausted into the environment. Examples of industrial flue gases include gases produced during ferrous metal products manufacturing, non-ferrous products manufacturing, petroleum refining processes, gasification of coal, gasification of biomass, electric power production, carbon black production, ammonia production, methanol production and coke manufacturing. The invention is also applicable to reactions which produce alternative alcohols.

Processes for the production of ethanol and other alcohols from gaseous substrates are known. Exemplary processes include those described for example in WO2007/117157, WO2008/115080, WO2009/022925, U.S. Pat. No. 6,340,581, U.S. Pat. No. 6,136,577, U.S. Pat. No. 5,593,886, U.S. Pat. No. 5,807,722 and U.S. Pat. No. 5,821,111, each of which is incorporated herein by reference.

A number of anaerobic bacteria are known to be capable of carrying out the fermentation of CO to alcohols, including n-butanol and ethanol, and acetic acid, and are suitable for use in the process of the present invention. Examples of such bacteria that are suitable for use in the invention include those of the genus Clostridium, such as strains of Clostridium ljungdahlii, including those described in WO 00/68407, EP 117309, U.S. Pat. Nos. 5,173,429, 5,593,886, and 6,368,819, WO 98/00558 and WO 02/08438, Clostridium carboxydivorans (Liou et al., International Journal of Systematic and Evolutionary Microbiology 33: pp 2085-2091) and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, et al (1991), Systematic and Applied Microbiology 14: 254-260). Further examples include Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Simpa et. al. Critical Reviews in Biotechnology, 2006 Vol. 26. Pp 41-65). In addition, it should be understood that other acetogenic anaerobic bacteria can be applicable to the present invention as would be understood by a person of skill in the art. It will also be appreciated that the invention may be applied to a mixed culture of two or more bacteria.

One exemplary micro-organism suitable for use in the present invention is Clostridium autoethanogenum. In one embodiment, the Clostridium autoethanogenum is or has the identifying characteristics of the strain deposited at the German Resource Centre for Biological Material (DSMZ) under the identifying deposit number 19630. In another embodiment, the Clostridium autoethanogenum is or has the identifying characteristics of DSMZ deposit number DSMZ 10061.

Culturing of the bacteria used in the methods of the invention may be conducted using any number of processes known in the art for culturing and fermenting substrates using anaerobic bacteria. Exemplary techniques are provided in the “Examples” section below. By way of further example, those processes generally described in the following articles using gaseous substrates for fermentation may be utilised: (i) Klasson, et al. (1991). Bioreactors for synthesis gas fermentations resources. Conservation and Recycling, 5; 145-165; (ii) Klasson, et al. (1991). Bioreactor design for synthesis gas fermentations. Fuel. 70. 605-614; (iii) Klasson, et al. (1992). Bioconversion of synthesis gas into liquid or gaseous fuels. Enzyme and Microbial Technology. 14; 602-608; (iv) Vega, et al. (1989). Study of Gaseous Substrate Fermentation: Carbon Monoxide Conversion to Acetate. 2. Continuous Culture. Biotech. Bioeng. 34. 6. 785-793; (vi) Vega, et al. (1989). Study of gaseous substrate fermentations: Carbon monoxide conversion to acetate. 1. Batch culture. Biotechnology and Bioengineering. 34. 6. 774-784; (vii) Vega, et al. (1990). Design of Bioreactors for Coal Synthesis Gas Fermentations. Resources, Conservation and Recycling. 3. 149-160; all of which are incorporated herein by reference.

The fermentation can be carried out in any suitable bioreactor, such as a continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR). Also, in some embodiments of the invention, the bioreactor may comprise a first, growth reactor in which the micro-organisms are cultured, and a second, fermentation reactor, to which fermentation broth from the growth reactor is fed and in which most of the fermentation product (e.g. ethanol and acetate) is produced.

According to various embodiments of the invention, the carbon source for the fermentation reaction is preferably a gaseous substrate containing CO. The gaseous substrate may be a CO-containing waste gas obtained as a by-product of an industrial process, or from another source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

Alternatively, the CO-containing gaseous substrate may be sourced from the gasification of biomass. The process of gasification involves partial combustion of biomass in a restricted supply of air or oxygen. The resultant gas typically comprises mainly CO and H₂, with minimal volumes of CO₂, methane, ethylene and ethane. For example, biomass by-products obtained during the extraction and processing of foodstuffs such as sugar from sugarcane, or starch from maize or grains, or non-food biomass waste generated by the forestry industry may be gasified to produce a CO-containing gas suitable for use in the present invention.

The CO-containing substrate will typically contain a major proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present.

While it is not necessary for the gaseous substrate to contain any hydrogen, the presence of H₂ should not be detrimental to product formation in accordance with methods of the invention. In particular embodiments, the presence of hydrogen results in an improved overall efficiency of alcohol production. The gaseous substrate may also contain some CO₂ for example, such as about 1% to about 80% CO₂ by volume, or 1% to about 30% CO₂ by volume.

Typically, the carbon monoxide will be added to the fermentation reaction in a gaseous state. However, the methods of the invention are not limited to addition of the substrate in this state. For example, the carbon monoxide can be provided in a liquid. For example, a liquid may be saturated with a carbon monoxide containing gas and the saturated liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101, Number 3/October, 2002) could be used for this purpose.

It will be appreciated that for growth of the bacteria and CO-to-alcohol fermentation to occur, in addition to the CO-containing substrate gas, a suitable liquid nutrient medium will need to be fed to the bioreactor. A nutrient medium will contain vitamins and minerals sufficient to permit growth of the micro-organism used. Anaerobic media suitable for the fermentation of ethanol using CO as the sole carbon source are known in the art. For example, suitable media are described in U.S. Pat. Nos. 5,173,429 and 5,593,886 and WO 02/08438, WO2007/117157, WO2008/115080, WO2009/022925, WO2009/058028, WO2009/064200, WO2009/064201 and WO2009/113878, referred to above. The present invention provides a novel media which has increased efficacy in supporting growth of the micro-organisms and/or alcohol production in the fermentation process. This media will be described in more detail hereinafter.

The fermentation is carried out under appropriate conditions for the desired process to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. Suitable conditions are described in WO02/08438, WO2007/117157, WO2008/115080 and WO2009/022925.

The optimum reaction conditions will depend partly on the particular micro-organism used. However, in general, it is preferred that the fermentation be performed at pressure higher than ambient pressure. Operating at increased pressures allows a significant increase in the rate of CO transfer from the gas phase to the liquid phase where it can be taken up by the micro-organism as a carbon source for the production of ethanol. This in turn means that the retention time (defined as the liquid volume in the bioreactor divided by the input gas flow rate) can be reduced when bioreactors are maintained at elevated pressure rather than atmospheric pressure.

Also, since a given CO-to-ethanol conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

The benefits of conducting a gas-to-ethanol fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that the ethanol product is consumed by the culture.

### Product Recovery

The products of the fermentation reaction can be recovered using known methods. Exemplary methods include those described in WO2007/117157, WO2008/115080, WO2009/022925, U.S. Pat. No. 6,340,581, U.S. Pat. No. 6,136,577, U.S. Pat. No. 5,593,886, U.S. Pat. No. 5,807,722 and U.S. Pat. No. 5,821,111. However, briefly and by way of example only ethanol and/or 2,3-butandiol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is well known in the art.

Extractive fermentation procedures involve the use of a water-miscible solvent that presents a low toxicity risk to the fermentation organism, to recover the ethanol from dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

Acetate, which is produced as by-product in the fermentation reaction, may also be recovered from the fermentation broth using methods known in the art.

For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol—and acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used in the processes of the present invention. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described these patents describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially. In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth is adjusted as described above to enhance adsorption of acetic acid to the activated charcoal, the pH is re-adjusted to a similar pH to that of the broth in the fermentation bioreactor, before being returned to the bioreactor.

### Addition of Sulfur

In accordance with particular aspects of the invention, one or more inorganic sulfur compound is added to a fermentation reaction, which typically takes place in a bioreactor, in order to improve microbial growth and/or maintain or improve alcohol productivity. In particular embodiments, an inorganic sulfur source comprising one or more inorganic sulfur compound(s), such as a polysulfide solution, can be added to a fermentation liquid nutrient media in a bioreactor, prior to the bioreactor being inoculated with an anaerobic microbial culture. Additionally, or alternatively, the inorganic sulfur source can be provided at predetermined time points or continuously following inoculation to maintain the concentration of one or more inorganic sulfur compound in the bioreactor within a predetermined concentration range. Those skilled in the art will appreciate suitable inorganic salts of sulfur suitable for use with the methods of the invention. In particular embodiments, the sulfur compound is substantially soluble in the fermentation broth and is preferably non-toxic to the culture. In particular embodiments, the one or more inorganic sulfur compound is a polysulfide solution such as sodium polysulfide and is provided at a desirable concentration from approx 0.1 mM sulfur to about 20 mM sulfur or about 1 mM sulfur to about 20 mM sulfur. In a particular embodiment, the sodium polysulfide is provided at a sulfur concentration of about 6 mM to about 18 mM. Those skilled in the art will appreciate the concentration of polysulfide and sulfur in a particular solution will depend on factors including how the polysulfide was synthesised, pH and temperature. However, those skilled in the art will appreciate methods for determining the concentration of polysulfide in solution. In certain embodiments, the polysulfide is provided at a concentration of approximately 3 mM, 6 mM or 18 mM sulfur.

The characteristics of the polysulfide may alter depending on the method of preparation. For example, polysulfides prepared from sulfur and sulfide may vary depending on the ratio of reactants. In particular embodiments, the polysulfide is prepared by mixing sodium sulfide and sulfur in a 5:1, or a 4:1, or a 3:1, or a 2:1 ratio.

It will be appreciated that concentrations of sulfur may need to be elevated as biomass increases. Accordingly, in particular embodiments, the one or more inorganic sulfur compound is provided such that the concentration of sulfur is maintained from about 1 mmol to about 20 mmol per gram of cells. In one embodiment, polysulfide is provided such that the concentration of sulfur is maintained at about 3 mmol per gram of cells, or 6 mmol per gram of cells, or 18 mM per gram of cells.

In particular embodiments of the invention, an inorganic sulfur source is provided, such that when the inorganic sulfur source is added to an aqueous nutrient media, one or more sulfur containing species are produced and are available to support microbial growth and/or product production. In particular embodiments, the sulfur containing species is at least one polysulfide species (such as Sₓ²⁻) and/or a sulfide species (such as HS⁻, S²⁻) and/or colloidal sulfur.

In particular embodiments of the invention, an inorganic sulfur source is provided such that when the one or more inorganic sulfur compound is added to an aqueous nutrient media, one or more sulfur containing species is released in a substantially continuous manner over a time period and is available to support growth and/or alcohol productivity of a microbial culture during that time period. In particular embodiments, the one or more inorganic sulfur compound can be added initially, at predetermined time points, or continuously in order to maintain a desired concentration of sulfur species in the media. Those skilled in the art will appreciate that particular inorganic sulfur sources may decompose and/or release one or more sulfur species at different rates depending on variables such as pH, temperature, pressure etc. Accordingly, as may be necessary to support growth and/or alcohol productivity of a microbial culture, additional inorganic sulfur compound(s) may be added in order to increase the concentration of the desirable sulfur species in solution.

It has been surprisingly recognised that addition of inorganic sulfur to a fermentation media comprising one or more carboxydotrophic micro-organisms results in improved microbial growth. In particular embodiments of the invention, wherein polysulfide is the inorganic sulfur source, carboxydotrophic bacteria, such as Clostridium autoethanogenum grow to a higher microbial density than when traditional sulfur sources such a cysteine are used. In particular embodiments, the growth rate of the microbial culture is also enhanced.

Additionally or alternatively, the provision of inorganic sulfur results in improved metabolite production. In particular embodiments of the invention, a microbial culture produces substantially more alcohol than would be typically produced by a microbial culture supplied with a traditional sulfur source, such as cysteine. In accordance with the methods of the invention, the one or more inorganic sulfur compound is added to a microbial culture such that a substrate comprising CO is converted to products including alcohols such as ethanol and/or 2,3-butanediol. Surprisingly, addition of inorganic sulfur to fermentation media can enhance microbial growth and/or alcohol production. Typically, alcohol, such as ethanol is produced concomitantly with acids, such as acetate, during fermentation of CO. However, in accordance with the methods of the invention, the alcohol to acid product ratio is at least 10:1; or at least 20:1; or at least 50:1; or at least 100:1 favouring alcohol. In particular embodiments, alcohol is produced without concomitant acids production.

In particular embodiments, 2,3-butandiol is produced in addition to ethanol. For example, fermentation of a substrate comprising CO by C. auto, ethanol and 2,3-butanediol are typically produced in a ratio of at least 10:1 favouring ethanol. However, in accordance with particular methods of the invention, 2,3-butanediol is provided such that the ethanol:2,3-butanediol ratio is less than 10:1, or less than 8:1, or less than 5:1, or less than 3:1, or less than 2:1. In accordance with particular embodiments of the invention, the rate at which alcohols including ethanol and/or 2,3-butanediol are produced in increased.

In certain embodiments, the sulfur source comprises a composition adapted to release one or more sulfur species, such as sulfide and/or colloidal sulfur, into a microbial culture over a predetermined time period. For example, a sulfur species can be physically and/or chemically captured in or on a capture means, said capture means being adapted to release the sulfur species over time. In addition to the polysulfide described herein, other examples of such carrier means include metal complexes, molecular sieves or other substantially porous materials. Carriers suitable for capturing CO are described in detail in PCT/NZ2008/000306 which is incorporated herein by reference. Such capture means may be adapted to capture and release hydrogen sulfide over predetermined time periods at concentrations sufficient to promote growth and/or alcohol productivity in a microbial culture.

### Nutrient Media

In particular embodiments of the invention, a novel fermentation media is provided which comprises one or more inorganic sulfur compound at a concentration of from approximately 0.1 mM to 20 mM sulfur. In particular embodiments, the media comprises polysulfide at a concentration of from approximately 0.1 mM to 20 mM sulfur. Persons of ordinary skill in the art to which the invention relates will readily appreciate the forms of polysulfide suitable for use in such a nutrient medium and the amount of sulfide and sulfur required to prepare the media on the basis of the desired concentration. However, by way of example, sodium polysulfide (Na₂Sₓ) is a suitable form for use in the methods of the invention.

In certain embodiments of the invention, a novel nutrient media is provided which comprises iron at a concentration of from approximately 16 mg/l to 280 mg/l, zinc at a concentration of from approximately 0.45 mg/l to 32 mg/l, manganese at a concentration of from approximately 1.6 mg/l to 27 mg/l, copper at a concentration of from approximately 0.1 mg/l to 100 mg/l, boron at a concentration of from approximately 1 mg/l to 5 mg/l, molybdenum at a concentration of from approximately 0.2 mg/l to 47 mg/l, selenium at a concentration of from approximately 0.1 mg/l to 39 mg/l, vanadium at a concentration of from approximately 0.5 mg/l to 50 mg/l, cobalt at a concentration of from approximately 1 mg/l to 147 mg/l, nickel at a concentration of from approximately 0.1 mg/l to 83 mg/l and tungsten at a concentration of from approximately 1.1 mg/l to 91 mg/l. It will be appreciated that the media will be prepared using one or more salts of these metals. Persons of ordinary skill in the art to which the invention relates will readily appreciate the amount of each salt required to prepare the media on the basis of the desired concentration of a particular metal and the molecular weights thereof.

In particular embodiments of the invention, a precipitate (typically black) is formed on addition of the polysulfide solution to the liquid nutrient medium containing various metal salts. The precipitate is attributed to at least one poorly soluble metal sulfide salt, such as FeS, ZnS, CoS and NiS. In particular embodiments, the presence of the precipitate during inoculation improves growth and/or productivity of the microbial culture following inoculation. Without wishing to be bound by theory, it is considered the precipitate particles provide a protection for the freshly inoculated microbial cells, such that they continue to metabolise after inoculation. Without the presence of the precipitate, the inoculated culture lags for several days before growth and/or product metabolism initiates. As such, in particular embodiments, a precipitate comprising at least one metal sulfide improves the efficiency of a fermentation of a substrate comprising CO. In particular embodiments, the precipitate is produced by adding polysulfide to the media. On (partial) decomposition, the resultant sulfide precipitates with metal ions present in the media, to produce the precipitate. In other embodiments, the precipitate can be formed separately, by adding sulfide, such as sodium sulfide to an aqueous metal solution. The resultant precipitate can be added to the liquid nutrient media, prior to inoculation.

It will be appreciated that the nutrient media may also contain other ingredients which are required or preferred for bacterial growth, as will be known in the art. Exemplary ingredients include those detailed herein after in the section entitled “Examples”. Further examples are provided in U.S. Pat. Nos. 5,173,429 and 5,593,886 and WO 2002/08438, WO2007/117157, WO2008/115080, WO2009/022925, WO2009/058028, WO2009/064200, WO2009/064201 and WO2009/113878, referred to above.

The media may be prepared in accordance with standard procedures known in the art, as exemplified herein and in U.S. Pat. Nos. 5,173,429 and 5,593,886 and WO 2002/08438, WO2007/117157, WO2008/115080, WO2009/022925, WO2009/058028, WO2009/064200, WO2009/064201 and WO2009/113878. Prior to use, if required, the media can be made anaerobic using standard procedures as exemplified herein in the section headed “Examples”.

### General

In accordance with the methods of the invention, components such as one or more inorganic sulfur compound and/or one or more metals may be added in salt form. Typically, the components will be added in the form of a composition in which one or more of the metals are present in combination with one or more diluents, carriers and/or other ingredients. In the simplest form of the invention, the composition consists of one or more inorganic sulfur compound and optionally the one or more of the metal ions/salts and water. In particular embodiments of the invention the one or more inorganic sulfur compound and the metal ions are provided in nutrient media substantially matching that in the fermentation reaction (other than a relatively higher concentration of the particular metal(s) compared to other ingredients), or an alternative complementary media. The composition is preferably buffered, for example, using sodium acetate buffered to approximately pH 5.5.

Solutions containing the desired components may be sterilized by autoclaving or filtration before being treated with anaerobic gas or transferred into an anaerobic atmosphere in order to remove the oxygen from these solutions.

The one or more inorganic sulfur compound and optionally one or more metals may be added to the fermentation reaction at any point at which an operator is concerned that the components are depleted to the point that they may be limiting the growth rate of the micro-organisms and/or alcohol production rate or otherwise to ensure the concentration of each remains in the range mentioned herein before. The components may be added at discrete time points or continuously fed to the bioreactor at a rate calculated to ensure that the concentration of the components in the broth are maintained within the range above mentioned. Persons of general skill in the art will be able to calculate an appropriate rate of continuous delivery of one or more components during the fermentation process based on observation of the average rates of growth of bacteria and/or alcohol production rate and the average rates of depletion of each component, particularly having regard to the “Examples” provided herein.

As to addition of components at discrete time points, one could determine average rates of microbial growth and/or alcohol production rate and depletion of the relevant components as mentioned above and calculate points in the fermentation reaction at which it is most likely metals and/or one or more inorganic sulfur compound will be required to be supplemented. Alternatively, an operator could actively monitor the fermentation process by taking samples from the bioreactor at particular time points to determine the levels of sulfur, manganese, zinc, copper, boron, molybdenum, selenium, vanadium, tungsten, iron, cobalt and/or nickel, and/or cell density, and/or alcohol productivity. Alternatively, or in addition, the concentration of particular components of the media may be continuously monitored by other means, such as one or more probes. The information gathered from such sampling would allow an operator to make an informed decision about the steps to take next. For example, if the level or concentration of one or more of sulfur, manganese, zinc, copper, boron, molybdenum, selenium, vanadium, tungsten, iron, cobalt and/or nickel was low (for example on the lower side of the preferred range provided herein before), and/or the cell density is low, the operator may decide to introduce one or more of the components to the bioreactor. If the concentration of one or more of the components was on the higher side of the range, or the cell density was desirable, the operator could choose to delay supplementing the fermentation reaction.

Levels of each of sulfur, manganese, zinc, copper, boron, molybdenum, selenium, vanadium, tungsten, iron, cobalt and nickel can be measured using methodology standard in the art. However, by way of example mass spectroscopy, inductively coupled plasma mass spectroscopy, HPLC, ion exchange chromatography, atomic absorption spectroscopy, atomic absorption mass spectroscopy or voltammetry could be used.

Cell density may be measured using standard techniques known in the art. However, by way of example, manual observation under a microscope or measurement of optical density using a spectrophotometer may be used. Measurement of optical density at 600 nm is a standard technique.

The components may be added to the bioreactor by any known means. However, by way of example solutions may be introduced into the reactor automatically through a dedicated pump or manually via septum covered port using a syringe.

### Metal Reducing Agents

It is well known that anaerobic fermentation such as fermentation of a substrate comprising CO, is typically conducted at low redox potential (ORP). For example the anaerobic fermentation of substrates comprising CO to products, such as acetate and/or alcohol, are typically conducted such that the ORP of the fermentation broth is less than −400 mV, or less than −450 mV, or less than −500 mV. In accordance with the methods of the invention, one or more metal reducing agents are added to an anaerobic microbial culture in order to attain and/or maintain a fermentation broth ORP sufficiently low to support microbial growth and/or alcohol production.

The ORP of the liquid nutrient medium of a microbial culture adapted to ferment CO to products, such as Clostridium autoethanogenum, is typically adjusted to around −200 mV prior to inoculation. Following inoculation, the microbial culture will typically self-regulate the ORP as the culture begins to grow, wherein the ORP typically decreases to around −400 mV to −450 mV. In particular aspects of the invention, one or more metal reducing agents, such as Cr(II) can be added to the liquid nutrient media, wherein the ORP of the liquid nutrient media can be decreased to less than −150 mV, or less than −200 mV, or less than −250 mV, or less than −300 mV, or less than −350 mV, or less than −400 mV, or less than −450 mV, or less than −500 mV, prior to inoculation with a microbial culture.

As noted previously, it is well known that fermentation products such as acetic acid, can be reduced to products such as alcohols by lowering the ORP of the fermentation broth. According to an additional or alternative aspect of the invention, one or more metal reducing agents such as Cr(II) can be added to a microbial culture in order to provide an ORP sufficiently low to promote alcohol production. In particular embodiments, Cr(II) is added to the fermentation broth such that the ORP is maintained at less than −400 mV, or less than −450 mV, or less than −500 mV.

It will be recognised by those skilled in the art upon consideration of the instant disclosure, that any metal reducing agent may be provided to reduce the ORP of the liquid nutrient media and/or fermentation broth, providing the metal is not toxic to the culture (in either reduced or oxidised form). In particular embodiments of the invention, the metal may be selected from V(II), Ti(III) and Cr(II). In particular embodiments, Cr(II) is used as the reducing agent as it is stable and reacts with oxygen substantially faster than other common metal reducing agents such as Ti(III).

In particular embodiments of the invention, Cr(II) may be added to a liquid nutrient media in order to attain a media ORP of approximately −150 mV to −200 mV prior to inoculation with Clostridium autoethanogenum. In particular embodiments, the liquid nutrient media comprises essential components such as metal ions and B vitamins required for microbial growth and/or alcohol production. In one embodiment of the invention, a sulfur source such as cysteine and/or H2S can be added to the reduced media prior to inoculation.

In certain embodiments, one or more metal reducing agent(s), such as Cr(II), may be added to the fermentation broth after inoculation such that the ORP of the fermentation broth is substantially maintained at a desirable level, such as less than −400 mV, or less than −450 mV, or less than −500 mV, or less than −550 mV, or less than −600 mV. Additionally or alternatively, additional liquid nutrient media reduced with one or more metal reducing agent(s), such as Cr(II), can be added to maintain the fermentation broth at a predetermined level. This may be beneficial in embodiments where large quantities of fresh media are continuously fed to a fermentation reaction, such as continuous fermentation.

In accordance with the methods of the invention, metal reducing agents, such as Cr(II), are typically added to a liquid nutrient media and/or fermentation broth in the form of an aqueous salt. However, it will be appreciated that in particular embodiments, it may be possible to provide the metal salt as a solid or substantially pure liquid. In particular embodiments of the invention, the metal reducing agent is provided as aqueous CrCl₂.

As noted previously, the one or more metal reducing agents may be added to the liquid nutrient media prior to inoculation, such that the ORP of the liquid nutrient media decreases to a predetermined desirable level to support microbial growth following inoculation. Additionally or alternatively, the metal reducing agent may be added to the fermentation broth at any point at which an operator is concerned that the ORP has increased above a desirable or predetermined level. For example, a predetermined amount of the metal reducing agent may be added to the fermentation broth such that the ORP of the fermentation broth decreases to the predetermined or desirable level/range. Additionally or alternatively, aliquots of the metal reducing agent may be added until the ORP of the broth reduces to the required level/range.

The one or more metal reducing agents may be added at one or more discrete time points or continuously fed to a bioreactor containing a fermentation reaction, at a rate calculated to ensure the ORP of the broth is maintained within the predetermined range. Persons of general skill in the art will be able to calculate an appropriate rate of continuous delivery of one or more components during the fermentation process based on the average ORP of the fermentation broth during microbial growth and/or alcohol production, particularly having regard to the “Examples” provided herein.

### EXAMPLES

### Media

Solution A

| | | | | | |
|---|---|---|---|---|---|
| NH₄Ac | 3.083 | g | KCl | 0.15 | g |
| MgCl₂•6H₂O | 0.61 | g | NaCl (optional) | 0.12 | g |
| CaCl₂•2H₂O | 0.294 | g | Distilled Water | Up to 1 | L |

Solution B

| | | | | | |
|---|---|---|---|---|---|
| Biotin | 20.0 | mg | Calcium D-(*)- | 50.0 | mg |
| | | | pantothenate | | |
| Folic acid | 20.0 | mg | Vitamin B12 | 50.0 | mg |
| Pyridoxine•HCl | 10.0 | mg | p-Aminobenzoic acid | 50.0 | mg |
| Thiamine•HCl | 50.0 | mg | Thioctic acid | 50.0 | mg |
| Riboflavin | 50.0 | mg | Distilled water | To 1 | Litre |
| Nicotinic acid | 50.0 | mg | | | |

Solution(s) C

| Component/0.1M | Quantity/ml into | Component/0.1M | Quantity/ml |
|---|---|---|---|
| solution (aq) | 1 L media | solution (aq) | into 1 L media |
| FeCl₃ | 10 ml | MnCl₂ | 1 ml |
| CoCl₂ | 5 ml | Na₂WO₄ | 1 ml |
| NiCl₂ | 5 ml | ZnCl₂ | 1 ml |
| H₃BO₃ | 1 ml | Na₂SeO₃ | 1 ml |
| Na₂MoO₄ | 1 ml | | |

Solution(s) D

| Component/0.1M solution (aq) | Quantity/ml into 1 L media |
|---|---|
| FeCl₂ | 10 ml |
| CoCl₂ | 10 ml |
| NiCl₂ | 10 ml |

### Preparation of Na₂Sₓ

A 500 ml flask was charged with Na₂S (93.7 g, 0.39 mol) and 200 ml H₂O. The solution was stirred until the salt had dissolved and sulfur (25 g, 0.1 mol) was added under constant N₂ flow. After 2 hours stirring at room temperature, the “Na₂Sₓ” solution (approx 4M with respect to [Na] and approx 5M with respect to sulfur), now a clear reddish brown liquid, was transferred into N₂ purged serum bottles, wrapped in aluminium foil.

### Preparation of Cr (II) Solution

A 1 L three necked flask was fitted with a gas tight inlet and outlet to allow working under inert gas and subsequent transfer of the desired product into a suitable storage flask. The flask was charged with CrCl₃.6H₂O (40 g, 0.15 mol), zinc granules [20 mesh] (18.3 g, 0.28 mol), mercury (13.55 g, 1 mL, 0.0676 mol) and 500 mL of distilled water. Following flushing with N₂ for one hour, the mixture was warmed to about 80° C. to initiate the reaction. Following two hours of stirring under a constant N₂ flow, the mixture was cooled to room temperature and continuously stirred for another 48 hours by which time the reaction mixture had turned to a deep blue solution. The solution was transferred into N₂ purged serum bottles and stored in the fridge for future use.

### Bacteria:

The Clostridium autoethanogenum used is that deposited at the German Resource Centre for Biological Material (DSMZ) and allocated the accession number 19630.

### Sampling and Analytical Procedures

Media samples were taken from the CSTR reactor at intervals over periods up to 10 days. Each time the media was sampled care was taken to ensure that no gas was allowed to enter into or escape from the reactor.

### HPLC:

HPLC System Agilent 1100 Series. Mobile Phase: 0.0025N Sulfuric Acid. Flow and pressure: 0.800 mL/min. Column: Alltech IOA; Catalog #9648, 150×6.5 mm, particle size 5 μm. Temperature of column: 60° C. Detector: Refractive Index. Temperature of detector: 45° C.

### Method for Sample Preparation:

400 μL of sample and 50 μL of 0.15M ZnSO₄ and 50 μL of 0.15M Ba(OH)₂ are loaded into an Eppendorf tube. The tubes are centrifuged for 10 min. at 12,000 rpm, 4° C. 200 μL of the supernatant are transferred into an HPLC vial, and 5 μL are injected into the HPLC instrument.

### Headspace Analysis:

Measurements were carried out on a Varian CP-4900 micro GC with two installed channels. Channel 1 was a 10 m Mol-sieve column running at 70° C., 200 kPa argon and a backflush time of 4.2 s, while channel 2 was a 10 m PPQ column running at 90° C., 150 kPa helium and no backflush. The injector temperature for both channels was 70° C. Runtimes were set to 120 s, but all peaks of interest would usually elute before 100 s.

### Cell Density:

Cell density was determined by counting bacterial cells in a defined aliquot of fermentation broth. Alternatively, the absorbance of the samples was measured at 600 nm (spectrophotometer) and the dry mass determined via calculation according to published procedures.

### Metal Sulfide Solution 1:

Approximately 950 mL of solution A was transferred into a 1 L fermenter and sparged with nitrogen. H₃PO₄ (85% solution, 1.5 mL, 30 mM) was added and the pH adjusted to 5.3 using conc NH₄OH(aq). Solution B (10 mL) was added and the solution sparged with N2. Chromium(II)chloride was added until the ORP of the solution decreased to approximately −200 mV, wherein resazurin (1 mL of a 2 g/L solution) was added. Solution(s) C then sodium polysulfide (2 mL of a 3M solution) were added and the solution sparged with N2

### Metal Sulfide Solution 2:

Approximately 950 mL of solution A (without NaCl) was transferred into a 1 L fermenter and sparged with nitrogen. H₃PO₄ (85% solution, 1.5 mL, 30 mM) was added and the pH adjusted to 5.3 using conc. NH₄OH(aq). Solution B (10 mL) was added and the solution sparged with N2. Chromium(II)chloride was added until the ORP of the solution decreased to approximately −200 mV, wherein resazurin (1 mL of a 2 g/L solution) was added. Solution(s) C then sodium polysulfide (2 mL of a 3M solution) were added and the solution sparged with N2

### Metal Sulfide Solution 3:

Approximately 9.6 g Na₂S.9H₂O was dissolved in approximately 70 ml H₂O and sparged with N2. Solution D was added and the pH adjusted to 6 with 1M HCl.

### Example 1A

### Batch Fermentation in CSTR Using Novel Nutrient Media and Polysulfide

Approximately 1.3 L of solution A was transferred into a 2 L fermenter and sparged with nitrogen. H₃PO₄ (85% solution, 2.025 mL, 30 mM) was added and the pH adjusted to 5.3 using conc. NH₄OH(aq). Solution B (13.5 mL) was added and the solution sparged with N2. Chromium(II)chloride was added until the ORP of the solution decreased to approximately −200 mV, wherein resazurin (1.35 mL of a 2 g/L solution) was added. Sodium polysulfide (2.7 mL of a 3M solution) was added and the solution sparged with N2 then CO containing gas (1% H2; 13% N2; 71% CO; 15% CO2). Metal sulfide solution 1 (150 mL) was added and the solution sparged a further 30 minutes, before inoculation with an actively growing Clostridium autoethanogenum culture at a level of approximately 5% (v/v).

### Example 1B

### Batch Fermentation in CSTR Using Novel Nutrient Media and Polysulfide

Approximately 900 ml of solution A was transferred into a 1 L fermenter and sparged with N2. H₃PO₄ (85% solution, 1.425 mL, 30 mM) was added and the pH adjusted to 5.3 using conc. NH₄OH(aq). Solution B (9.5 mL) was added and the solution sparged with N2. Chromium(II)chloride was added until the ORP of the solution decreased to approximately −150 mV, wherein resazurin (0.95 ml of a 2 g/L solution) was added. Sodium polysulfide (3.8 mL of a 3M solution) was added and the solution sparged with N2 then CO containing gas (1% H2; 13% N2; 71% CO; 15% CO2). Metal sulfide solution 1 (50 mL) was added and the solution sparged a further 30 minutes, before inoculation with an actively growing Clostridium autoethanogenum culture at a level of approximately 5% (v/v).

### Example 1C

### Batch Fermentation in CSTR Using Novel Nutrient Media and Polysulfide

Approximately 1.3 L of solution A was transferred into a 2 L fermenter and sparged with nitrogen. H₃PO₄ (85% solution, 2.025 mL, 30 mM) was added and the pH adjusted to 5.3 using conc. NH₄OH(aq). Solution B (13.5 mL) was added and the solution sparged with N2. Chromium(II)chloride was added until the ORP of the solution decreased to approximately −200 mV, wherein resazurin (1.35 mL of a 2 g/L solution) was added. Sodium polysulfide (5.4 mL of a 3M solution) was added and the solution sparged with N2 then CO containing gas (1% H2; 13% N2; 71% CO; 15% CO2). Metal sulfide solution 1 (150 mL) was added and the solution sparged a further 30 minutes, before inoculation with an actively growing Clostridium autoethanogenum culture at a level of approximately 5% (v/v).

Results:

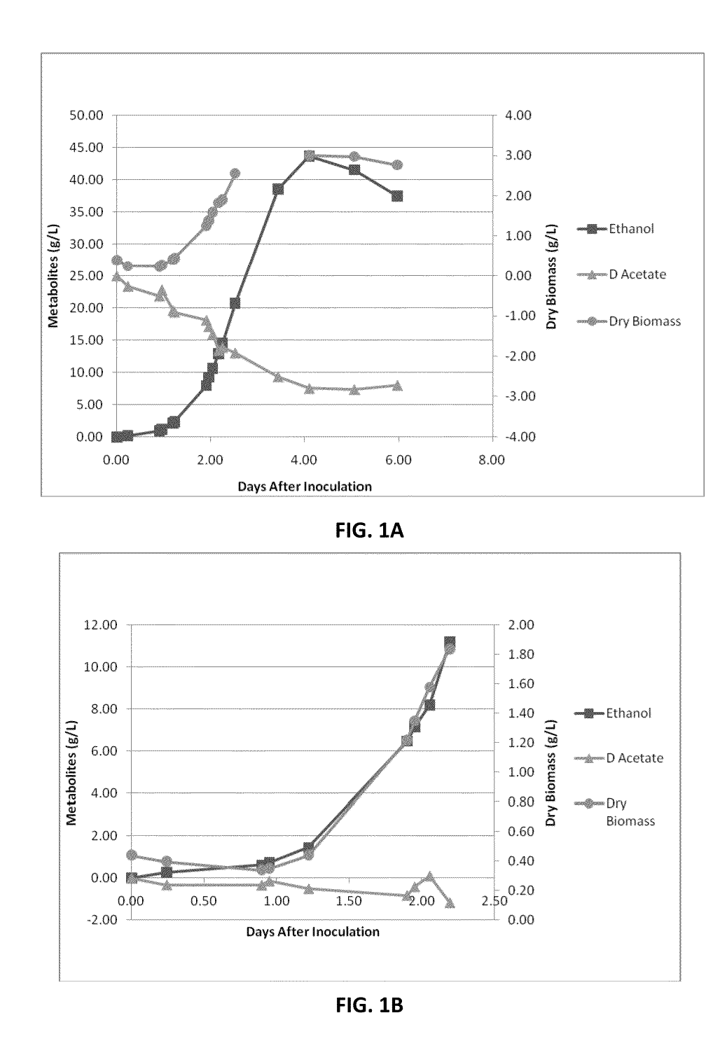

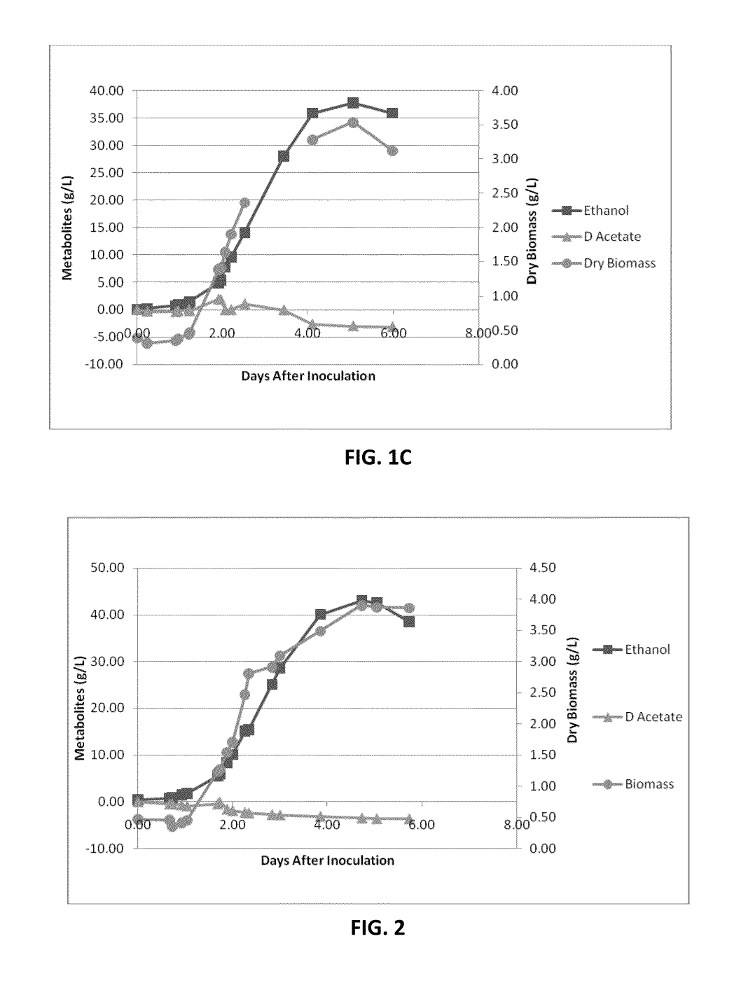

FIGS. 1A, B and C show microbial growth and ethanol productivity against time in batch fermentation experiments under similar conditions. FIG. 1A shows the results from example 1A, FIG. 1B shows the results from example 1B and FIG. 1C shows the results from example 1C. Fermentation of a substrate comprising CO by C. autoethanogenum under batch conditions typically results in a biomass accumulation of approximately 1.0-1.2 g/L over a 2-3 day period. However, in the presence of polysulfide, the biomass can accumulate to much higher levels (approximately 4 g/L). Furthermore, the rate of accumulation is also substantially increased. For example, following a short lag phase of approximately 1 day, the biomass increases from approximately 0.5 g/L, up to at least 2 g/L, or at least 2.5 g/L, or at least 3 g/L, or at least 3.5 g/L over approximately 36 hours.

Furthermore, alcohol productivity is also increased. Typically, batch fermentation of CO by C. autoethanogenum typically produces acetate and alcohol to levels up to 15 g/L and 10 g/L respectively, over several days. However, in the examples, where polysulfide is provided at various concentrations, ethanol productivity increases to at least 10g/L/day, or at least 15 g/L/day, or at least 20 g/L/day over the growth period of the microbial culture. During typical batch fermentations, acetate is produced during the growth phase of the microbial culture. However, in the presence of polysulfide, acetate is not produced during the growth phase. Furthermore, in particular examples, some of the acetate is consumed, such that there is a net decrease in the amount of acetate in the fermenter(s).

### Example 2

### Batch Fermentation in CSTR Using Novel Nutrient Media and Polysulfide

Approximately 1.3 L of solution A (without NaCl) was transferred into a 2 L fermenter and sparged with nitrogen. Resazurin (1.35 mL of a 2 g/L solution) then H₃PO₄ (85% solution, 2.025 mL, 30 mM) were added and the pH adjusted to 5.3 using conc. NH₄OH(aq). Sparge with N2. Chromium(II)chloride was added until the ORP of the solution decreased to approximately −150 mV. Sodium polysulfide (6.07 mL of a 4.3M solution) was added and the solution sparged with N2 for 1 hour before adding metal sulphide solution 2 (150 mL). Solution B (15 mL) was added and the solution sparged with N2 then CO containing gas (3% H2; 30% N2; 47% CO; 2% CO2) before inoculation with an actively growing Clostridium autoethanogenum culture at a level of approximately 5% (v/v). Additional sodium polysulfide (6.07 mL of a 4.3M solution) was added on day 2.

Results:

FIG. 2 shows that under desirable conditions (up to 18 mM polysulfide added at start), the microbial culture can grow to at least 3.5 g/L (dry weight) over several days in a batch fermenter. Furthermore, alcohol productivity rates can reach over 20 g/L/day.

### Example 3

### Batch Fermentation in CSTR Using Novel Nutrient Media and Polysulfide

Approximately 1.5 L of solution A (without NaCl) was transferred into a 2 L fermenter and sparged with nitrogen. Resazurin (1.5 ml of a 2 g/L solution) then H₃PO₄ (85% solution, 2.25 ml, 30 mM) were added and the pH adjusted to 5.3 using conc. NH₄OH(aq). Chromium(II)chloride was added until the ORP of the solution decreased to approximately −150 mV. Sodium polysulfide (0.25 ml of a 3M solution containing approx 1% Se) and 1% Se was added and the solution sparged with N2. 0.1M solutions of FeCl₃ (1 mL), CoCl₂ (0.5 mL), NiCl₂ (0.5 mL), H₃BO₃ (0.5 mL), Na₂MoO₄ (0.1 mL), MnCl₂ (0.1 mL), Na₂WO₄ (0.1 mL) and ZnCl₂ (0.1 mL) were added. Metal sulphide solution 2 (10 mL), then solution B (30 ml) were added before switching to CO containing gas (2% H2; 30% N2; 49% CO; 19% CO2). The pH was adjusted to 5.5 before inoculation with an actively growing Clostridium autoethanogenum culture at a level of approximately 5% (v/v). A further 2.5 ml Sodium Polysulfide solution/day was pumped into the fermenter over 2 days. Then unfiltered polysulfide solution (10 mL of a 4.3M solution) was pumped into the fermenter over 3 days.

Results:

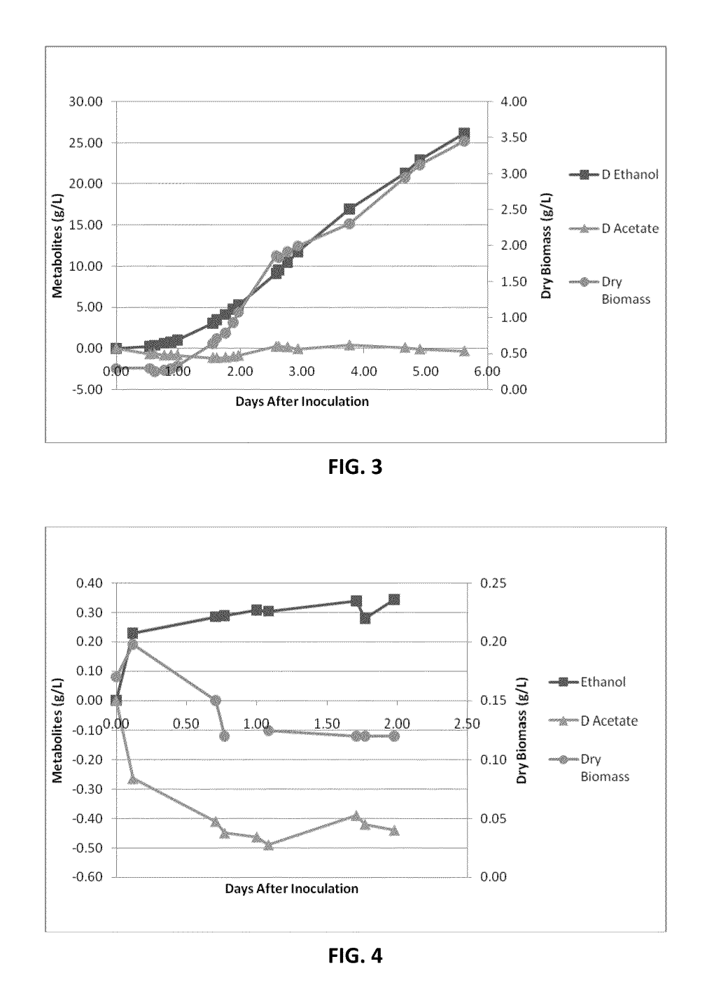

FIG. 3 shows biomass and metabolite accumulation in a batch fermentation where polysulfide was continuously added. Continuous addition of polysulfide over 2 days extended the growth phase of the culture, such that the microbial culture continued to grow for over 5 days. Again, ethanol is the primary metabolite formed without any concomitant acetate production.

### Example 4

### Batch Fermentation in CSTR Using Novel Nutrient Media and Polysulfide

Approximately 900 ml of solution A was transferred into a 1 L fermenter and sparged with nitrogen. H₃PO₄ (85% solution, 1.35 ml, 30 mM) was added and the pH adjusted to 5.3 using conc. NH₄OH(aq). Solution B (9 mL) was added and the solution sparged with N2. Chromium(II)chloride was added until the ORP of the solution decreased to approximately −150 mV, wherein resazurin (0.9 ml of a 2 g/L solution) was added. Sodium polysulfide (1.8 ml of a 3M solution) was added and the solution sparged with N2 and then CO containing gas (51% H2; 30% CO; 4% CO2; 14% CH4). Metal sulphide solution 1 (100 mL) was added through a filter and the solution sparged for a further 30 mins before inoculation with an actively growing Clostridium autoethanogenum culture at a level of approximately 5% (v/v).

Results:

FIG. 4 shows the initial stages of a batch fermentation, wherein the metal sulfide solution is filtered prior to addition to the fermenter. Filtration of the metal sulfide solution results in removal of all precipitate before it is added to the fermenter. There is a long lag phase (2 days), where the microbial culture does not grow and metabolites are not produced. It is considered that the metal sulfide precipitate provides protection for the freshly inoculated culture. In this example the black precipitate is filtered (removed) from the solution and the unprotected culture is slow to establish.

### Example 5

### Batch Fermentation in CSTR Using Novel Nutrient Media and Polysulfide

Approximately 1.5 L of solution A (minus NaCl) was transferred into a 2 L fermenter and sparged with nitrogen. Resazurin (1.5 ml of a 2 g/L solution) was added. H₃PO₄ (85% solution, 2.25 ml, 30 mM) was added and the pH adjusted to 5.3 using conc. NH₄OH(aq). Chromium(II)chloride was added until the ORP of the solution decreased to approximately −150 mV. Sodium polysulfide (6.28 ml of a 4.5M solution) was added and the pH adjusted to 5.5 with HCl. The solution was sparged with N2 for 1.5 hours. 1/10 solution C was added before switching to CO containing gas (3% H2; 30% N2; 50% CO; 20% CO2). 10 ml of metal sulphide solution 3 and 15 ml solution B was added before inoculation with an actively growing Clostridium autoethanogenum culture at a level of approximately 5% (v/v).

Results:

FIG. 5 shows biomass and metabolite accumulation in a batch fermentation, wherein a protective precipitate is added. In this example, the precipitate is formed by addition of sodium sulfide to a metal solution. The resultant solution containing a black precipitate is added to the fermenter prior to inoculation to provide a protective environment for establishment of the culture.

### Example 6

### Batch Fermentation in CSTR Using Novel Nutrient Media and Polysulfide

Approximately 1.5 L of solution A (minus NaCl) was transferred into a 2 L fermenter and sparged with nitrogen. Resazurin (1.5 ml of a 2 g/L solution) was added. H₃PO₄ (85% solution, 2.25 ml, 30 mM) was added and the pH adjusted to 5.3 using conc. NH₄OH(aq). Chromium(II)chloride was added until the ORP of the solution decreased to approximately −150 mV. Sodium polysulfide (6.28 ml of a 4.5M solution) was added and the pH adjusted to 5.5 with HCl. The solution was sparged with N2 for 1.5 hours. 1/10 solution C and 15 ml solution B was added before switching to CO containing gas (3% H2; 30% N2; 50% CO; 20% CO2). The reactor was inoculated with an actively growing Clostridium autoethanogenum culture at a level of approximately 5% (v/v).

Results:

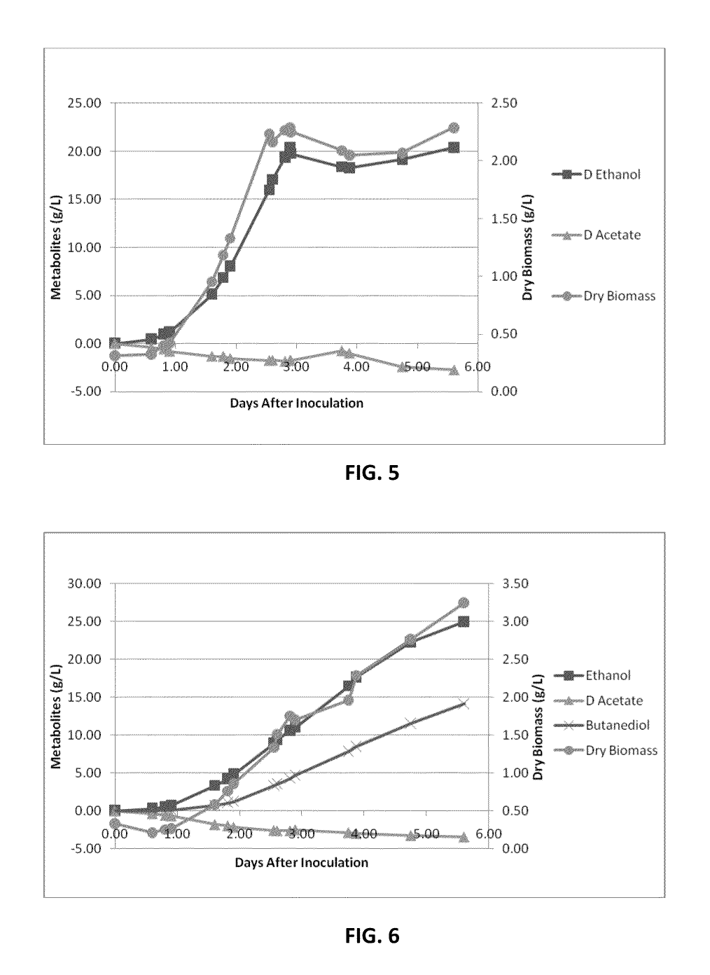

FIG. 6 shows biomass and metabolite accumulation in a batch fermentation of a substrate comprising CO. The media comprises polysulfide solution and 2,3-butanediol production is enhanced such that the enthanol:2,3-butanediol product ratio is less than 2:1.

The invention has been described herein with reference to certain preferred embodiments, in order to enable the reader to practice the invention without undue experimentation. Those skilled in the art will appreciate that the invention is susceptible to variations and modifications other than those specifically described. It is to be understood that the invention includes all such variations and modifications. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

What we claim is:

1. A method of producing one or more products by microbial fermentation, the method comprising:
(a) providing a gaseous substrate comprising carbon monoxide to a bioreactor comprising a culture of at least one carboxydotrophic micro-organisms in a liquid nutrient medium that comprises at least one inorganic sulfur compound, anaerobically fermenting the substrate to produce one or more products selected from the group consisting of acids, alcohols and mixtures thereof;
(b) recovering the one or more products produced; and wherein the concentration of the inorganic sulfur compound in the liquid nutrient medium is maintained at a concentration of about 6 mM to about 18 mM sulfur.

2. The method of claim 1 wherein the inorganic sulfur compound is releases one or more sulfur containing species into the liquid nutrient medium.

3. The method of claim 1 wherein the alcohol is ethanol and the acid is acetate, and wherein the ethanol-to-acetate ratio is at least 10:1.

4. The method of claim 1 wherein, the alcohol is ethanol and the acid is acetate and wherein the ethanol is substantially produced without concomitant acetate.

5. The method of claim 1 wherein the inorganic sulfur compound is provided as a polysulfide solution.

6. The method of claim 1 wherein the substrate comprising CO, comprises at least about 15% to about 100% by volume.

7. The method of claim 6 wherein the substrate comprising CO, comprises at least about 40% to at least about 70% CO by volume.

8. The method of claim 1 wherein the substrate comprises a gas obtained from a steel mill.

9. The method of claim 1 wherein the micro-organism is selected from the group consisting of Peptostreptococcus, Clostridium, Moorella, Pyrococcus, Eubacterium, Desulfobacterium, Carboxydothermus, Acetogenium, Acetobacterium, Acetoanaerobium, Butyribacterium and Peptostreptococcus.

10. The method of claim 9 wherein the micro-organism is Clostridium autoethanogenum.

11. The method of claim 1 wherein the substrate further comprises carbon dioxide and/or hydrogen.

12. The method of claim 1 wherein the microbial growth of the carboxydotrophic micro-organism is increased.

13. The method of claim 1 wherein the rate of production of at least one product is increased.

14. The method of claim 1 wherein the alcohol produced is ethanol and 2,3-butanediol at a ratio of less than 10:1 ethanol:2,3-butanediol.

15. The method of claim 1 wherein the liquid nutrient medium further comprises a compound of an element selected from the group consisting of Mn, Zn, B, Mo, Se, V, W, Fe, Co, Ni and mixtures thereof.

16. The method of claim 15 wherein the concentration of the elements in the liquid nutrient medium is within the range of about 0.3 mM to about 5 mM for Fe; about 7 μM to about 0.5 mM for Zn; about 30 μM to about 0.5 mM for Mn; about 0.1 mM to about 05 mM for B; about 17 μM to about 2.5 mM for Co; about 3 μM to about 2.5 mM for Ni; about 2.75 μM to about 0.5 mM for Mo; about 1.2 μM to about 0.5 mM for Se; about 6 μM to about 0.5 mM for W; and about 10 μM to about 1 mM for V.

17. A process for the anaerobic microbial fermentation of CO comprising:
a) providing a gaseous substrate comprising carbon monoxide to a bioreactor comprising a culture of at least one carboxydotrophic micro-organisms in a liquid nutrient medium that comprises at least one inorganic sulfur compound;
b) anaerobically fermenting the substrate to produce at least one product selected from the group consisting of acids, alcohols and mixtures thereof;
c) recovering at least one of the products;
d) maintaining the concentration of the inorganic sulfur compound in the liquid nutrient medium at level to provide from about 6 mM to about 18 mM sulfur thereby increasing the production of at least one of the products and increasing the microbial growth of the carboxydotrophic micro-organism.

18. The process of claim 17 wherein the alcohol is ethanol and the acid is acetate, and wherein the ethanol-to-acetate ratio is at least 10:1.

19. The process of claim 17 wherein the micro-organism is selected from the group consisting of Peptostreptococcus, Clostridium, Moorella, Pyrococcus, Eubacterium, Desulfobacterium, Carboxydothermus, Acetogenium, Acetobacterium, Acetoanaerobium, Butyribacterium and Peptostreptococcus.

20. The method of claim 19 wherein the micro-organism is Clostridium autoethanogenum.
